(19) United States
(12) Patent Application Publication
(10) Pub. No.: US 2024/0122208 A1
(43) Pub. Date: Apr. 18, 2024

(54) A PROCESS FOR PREPARING A VEGAN EDIBLE PRODUCT FROM EDIBLE NON-ANIMAL PROTEINS

(71) Applicant: BK GIULINI GMBH, Ladenburg (DE)

(72) Inventors: Wolfgang SCHNEIDER, Mannheim (DE); Alexander GRABER, Mannheim (DE); Andreas HEYL, Neustadt/Weinstrasse (DE); Radovan SPORKA, Heidelberg (DE)

(21) Appl. No.: 18/274,863

(22) PCT Filed: Feb. 9, 2022
(86) PCT No.: PCT/EP2022/053065

§ 371 Date: Jul. 28, 2023

(30) Foreign Application Priority Data
Feb. 10, 2021 (EP) 21156341.6

(51) Int. Cl. A23J 3/22; A23J 3/14; A23J 3/28; A23L 29/231; A23L 29/256; A23L 29/262

(52) U.S. Cl. CPC A23J 3/227; A23J 3/14; A23J 3/28; A23L 29/231; A23L 29/256; A23L 29/262

(57) ABSTRACT

The present invention relates to a process for preparing a vegan edible product from edible non-animal proteins which comprises the following steps i to iii.: (i) providing a malleable mass by mixing the following components: a) 7 to 20% by weight, in particular 10 to 18% by weight and especially 13 to 16% by weight, based on the total weight of the malleable mass, of an edible protein component A, which is selected from the group consisting of edible vegetable protein materials, microbial protein materials and mixtures thereof; b) 1 to 3.3% by weight, in particular 1.1 to 2.8% by weight, especially 1.2 to 2.3% by weight, based on the total weight of the malleable mass, of a water-soluble organic polymeric gelling agent which is capable of being gelled by calcium ions as a component B, which is a water-soluble polysaccharide bearing carboxyl groups or a water soluble salt thereof; c) optionally 0.05 to 1% by weight, in particular 0.1 to 0.9% by weight, especially 0.2 to 0.8% by weight, based on the total weight of the malleable mass, of a water-swellable nonionic polysaccharide as a component C; and d) 1 to 15% by weight, in particular 3 to 12% by weight, especially 5 to 10% by weight, based on the total weight of the malleable mass of an edible fat or oil of plant origin as a component D; e) water ad 100% by weight; (ii) comminuting the malleable mass into particles and (iii) bringing the particles into contact with an aqueous solution of a calcium salt to achieve a hardening of the particle, where step (iii) is carried out simultaneously with step (ii) or after step (ii). The thus obtained vegan edible products are suitable for preparing vegan artificial meat products.

The present invention relates to a process for preparing a vegan edible product from edible non-animal proteins which comprises
i. providing a malleable mass containing a vegetable and/or microbial protein material, a water-soluble gelling agent, which is capable of being gelled by calcium ions, a water-swellable nonionic polysaccharide, an edible fat or oil of plant origin and water
ii. comminuting the malleable mass into particles and
iii. bringing the particles into contact with an aqueous solution of a calcium salt to achieve a hardening of the particles.

The thus obtained edible products are suitable for preparing vegan artificial meat products.

As a basic principle, the main challenge of meat replacement is based on the fact that, with the exception of fibrous muscle meat, which in in its smallest units is predominantly composed of linear protein chains, there is no other protein that naturally forms such fibres.

It is principally known in the art to produce artificial meat products from proteins by a process, which comprises
(1) emulsifying the protein in the presence of a polysaccharide bearing carboxyl groups, such as alginate or pectin, with water and an oil or fat to obtain a viscous emulsion of the protein and the polysaccharide;
(2) comminuting or forming the resultant emulsion into particles, and
(3) simultaneously bringing the particles in contact with an aqueous solution of a bivalent metal salt, such as a water-soluble calcium salt, e.g. by soaking the particles in the aqueous solution of a bivalent metal salt.

Due to the hydration of the protein and the polysaccharide, the emulsion obtained in step (1) is a dough-like, malleable mass that can be comminuted and formed into particles, having the desired shape, in the presence of a bivalent metal salt, in particular a calcium salt. In step (3) the bivalent metal salt diffuses into the particles. Thereby, it causes a crosslinking of the polysaccharide and a precipitation/gelling of the protein/polysaccharide mixture resulting in a hardening of the shaped mass. The obtained mass can be further processed to artificial meat products.

Such a process is disclosed, for example, in EP 174192 A2, where a mass made of casein, an acidic polysaccharide and water is treated at an elevated temperature, followed by shaping the mass and soaking the mass in an aqueous solution of a multivalent metal salt. Modifications of said process, which cope with the specific requirements of the used milk protein, are described in WO 03/061400 and EP 1588626. As all these processes start from milk proteins, the final food products made therefrom could only be classified as vegetarian but not vegan.

NL 1008364 discloses the preparation of an artificial meat product containing no animal proteins comprises the following steps:
(a) preparation of a mixture of a non-animal protein, a plant-derived thickener capable of being precipitated/gelled with bivalent metal salts, such as pectin and alginate, and water;
(b) intensive stirring of the mixture at 40-90° C. to form an emulsion;
(c) mixing the emulsion with a salt solution containing a calcium and/or magnesium salt, to form a fibrous product, which is then further processed.

In this process, the fibre formation is controlled by the stirring speed when mixing the emulsion with salt solution. While the product obtained by this process can be classified as vegan, fibre formation is difficult to control and results in non-uniform fibre formation. Thus, the product quality may vary strongly. Moreover, only emulsions with low protein content were processed and thus, the process resulted in products having a low dry matter content and a low protein content. The product must therefore be pressed in order to increase the dry matter content.

EP 1790233 discloses a process for the preparation of an artificial meat product, where a protein and a fat are emulsified in water followed by subsequently incorporating a thickener, such as alginate, and a precipitant, such as calcium chloride into the emulsion. However, this process does not allow for precisely controlling the fibre structure, since the precipitation takes place very abruptly. Moreover, only small protein concentrations can be handled and thus a further separation step for removing the water from the precipitated emulsion is required.

WO 2014/111103 discloses a process for producing a meat substitute product, which comprises providing an emulsion of a mixture of an edible protein, such as caseinate or a plant protein, alginate, methyl cellulose, an oil and water, and precipitation of the emulsion by adding a combination of CaCl₂ and micellar casein. The amount of added CaCl₂ is chosen so that it alone is not sufficient to bring about complete precipitation. Rather, the use of micellar casein, which releases calcium ions in a controlled manner, enables a homogeneously precipitated fibre structure. The amount of added methyl cellulose affects the strength of the fibre which can be adjusted depending on the intended use. While the process allows for a better control of fibre formation, the protein and dry matter content of the fibres produced is comparatively low and protein contents of more than 10% and dry matter contents of more than 22% are difficult to obtain. Because of the use of micellar casein as a precipitant, the product can only be classified as vegetarian.

In contrast to caseinate and other animal proteins, which considerably contribute to an improvement of binding and texture, vegetable proteins and also most microbial proteins can be less well hydrated and therefore provide a poorer texture and structure. Therefore, it is more difficult for vegetable proteins than for animal proteins to achieve a sufficient, homogeneously precipitated fibre structure and to positively affect the sensorial perception with a similar succulence/moisture content as meat.

It is apparent from the foregoing that vegetable proteins and microbial proteins are more difficult to process to meat-substitute products than proteins of animal origin. In particular, fibre formation in the processes of prior art relating to vegetable proteins is either difficult to control or requires caseinate for achieving a good control of the fibre formation. Moreover, the processes do not allow for processing emulsions having a high content of proteins from plant or microbial origin. Rather the processes suggested so far for products based on plant proteins only achieve low dry matter and protein contents or require a further separation step to achieve an acceptable dry matter content. Simply increase the protein concentration in the emulsion to be processed does not overcome these problems, because modifying the known processes for producing meat-substitute products by processing emulsions containing the protein material in concentrations of 7% by weight or more will not result in a fibre material having an acceptable texture and do not provide sufficient, homogeneously precipitated fibre structure. Therefore, the processes do not allow to produce products solely from vegetable proteins, which positively affect the sensorial perception with a similar succulence/moisture content as meat.

It is therefore an object of the present invention to provide a process which overcomes the drawbacks of prior art. The process should allow for producing protein products based solely on non-animal, i.e. vegetable and/or microbial proteins, and thus protein products, which qualify as vegan products. In particular, the process should provide for a controllable and uniform formation of meat-like fibre and does not require the use of animal protein for the formation of the matrix or during precipitation. The process should yield products having a positive sensorial perception with a similar succulence/moisture content as meat. The process should be applicable and for many vegetable and microbial proteins and also allows for producing allergen-free products. Moreover, the process should be capable of providing edible protein products having a high protein content and still have the aforementioned benefits of good product quality. In particular, the process should provide these benefits, if it is carried out on an industrial scale, e.g. on a scale of tons per day or more. The process should be capable of being carried out in continuous and semi-continuous production processes.

It has been found that these objectives are met by the process which comprises the following steps (i) to (iii):
(i) providing a malleable mass by mixing the following components
   a) 7 to 20% by weight, in particular 8.5 to 18% by weight or 10 to 18% by weight and especially 13 to 16% by weight, based on the total weight of the malleable mass, of an edible protein component A, which is selected from the group consisting of edible vegetable protein materials, microbial protein materials and mixtures thereof,
   b) 1 to 3.3% by weight, in particular 1.1 to 2.8% by weight, especially 1.2 to 2.3% by weight, based on the total weight of the malleable mass, of a water-soluble organic polymeric gelling agent which is capable of being gelled by calcium ions as a component B, which is a water-soluble polysaccharide bearing carboxyl groups or a water soluble salts thereof,
   c) optionally 0.05 to 1% by weight, in particular 0.1 to 0.9% by weight, especially 0.2 to 0.8% by weight, based on the total weight of the malleable mass, of a water-swellable nonionic polysaccharide as a component C and
   d) 1 to 15% by weight, in particular 3 to 12% by weight, especially 5 to 10% by weight, based on the total weight of the malleable mass of an edible fat or oil of plant origin as a component D,
   e) water ad 100% by weight;
(ii) comminuting the malleable mass into particles and
(iii) bringing the particles into contact with an aqueous solution of a calcium salt to achieve a hardening of the particle, where step (iii) is carried out simultaneously with step (ii) or after step (ii).

Therefore, the present invention relates to a process for preparing a vegan edible product from an edible non-animal protein material, which comprises the steps i. to iii. as described herein.

The process allows for producing protein products based solely on non-animal protein materials, i.e. vegetable and/or microbial protein materials, with controllable and uniform formation of meat-like fibre and does not require the use of animal protein for the formation of the matrix or during precipitation and thus the protein can be classified as vegan. The process is not limited to particular vegetable proteins or microbial proteins and therefore allows for producing allergen-free products. Although the protein products obtained by the process of the invention are solely based on non-animal proteins, they have a positive sensorial perception with a similar succulence/moisture content as meat. Moreover, the process is capable of providing edible protein products having a high protein content and still have the aforementioned benefits of good product quality. In particular, the process provides these benefits, if it is carried out on an industrial scale, e.g. on a scale of 10 tons per day or more. The process is also capable of being carried out in continuous and semi-continuous production processes. Moreover, the process is less time consuming than the processes disclosed in prior art, as the time required for achieving an acceptable hardness is significantly smaller than in the process of prior art. Moreover, no time-consuming pressing step is required to achieve high protein and dry matter contents.

The invention is based on the surprising finding that a suitable mass ratio of a non-animal protein component A, in particular a vegetable protein component A, component B and component C is required to achieve a proper hydration of the protein component A, component B and component C, which is prerequisite for the above benefits. In contrast to prior art the process of the invention does not require animal proteins such as caseinate to achieve a controlled hardening and appreciable texture.

The process yields a particulate edible protein product, hereinafter also termed as protein fibre, which can be easily processed to an artificial meat product. Therefore, the present invention also relates to a process for preparing a vegan artificial meat product which comprises producing a vegan edible product from edible vegetable and/or microbial protein materials by the process as defined herein, followed by processing the vegan edible product to vegan artificial meat products. The processing can be carried out by analogy to the known methods of processing protein material to artificial meat products. The vegan edible products obtained by the process of the present invention can be used for producing vegan artificial meat products of any quality including vegan artificial meat products with a texture or mouthfeel comparable to meat or meat products from mammalian meat such as pork, beef, veal, lamb or goat, from poultry such as chicken, duck or goose, and products comparable to fish or seafood.

The invention is hereinafter explained in detail. Further embodiments can also be taken from the claims.

As the process relates to the production of edible products, a skilled person will immediately understand that all of the compounds and components, respectively, used for the production are edible constituents or at least are authorized additives for use in food, e.g. according to Regulation (EC) No 1333/2008 of the European Parliament and of the Council of 16 December 2008 on food additives. As the process is in particular directed to the preparation of a vegan edible product, a skilled person will immediately understand that all compounds and components, respectively, used in this process are in particular not of animal origin. In particular, no components of animal origin, such as animal protein components and animal fat, are used in this process. In particular, the process is carried out in the absence of any animal protein. Especially, the process is carried out in the absence of micellar casein in steps ii) and iii).

The term “edible protein material”, i. e. the component A, refers to a material highly enriched with edible protein, i.e. which typically has an analytical protein content of at least 70% by weight, in particular from 80 to 95% by weight in dry matter. The protein material of component a) is typically obtained by isolation from a natural, non-animal protein source, e.g. from a protein containing plant or a microorganism. Besides the protein, the protein material may contain other edible ingredients, such as carbohydrates and fats/oils contained in the protein source. Preferably, the edible protein material of component A is a protein isolate. Such a protein isolate generally has a protein content in the range of 80 to 95% in dry matter. The edible protein material of component A may also be a protein concentrate, which however, preferably has an analytical protein content of at least 70% by weight in dry matter.

Any amounts of component A in the malleable mass given here refer to the amount of component A as such.

The term “non-animal protein material” refers to any protein material from non-animal origin, i.e. to vegetable protein materials, microbial protein materials and mixtures thereof.

Here and in the following, the term “edible vegetable protein material” is an edible protein material from a vegetable source, i.e. from plants, which is suitable as food or food component for human nutrition.

Here and in the following, the term “edible microbial protein material” is an edible protein material from a microorganism source, i.e. from fungi, yeast or bacteria, which is suitable as food or food component for human nutrition. In this context protein from algae protein material may be considered both as a microbial protein material or as a vegetable protein material.

Here and in the following, the term “artificial meat product” includes any edible protein product produced from a non-animal protein material and having a texture or mouthfeel which is comparable to natural meat or products made from natural meat, including mammalian meat such as pork, beef, veal, lamb or goat, meat from poultry such as chicken, duck or goose, meat from fish or seafood.

The malleable mass contains a vegetable protein material or a microbial protein material or a mixture thereof, which is suitable for nutrition purposes, in particular for human nutrition. Hereinafter, the edible vegetable or microbial protein material is also referred to as component A or protein material. In particular, the protein material does not contain any protein of animal origin. Apart from that, the kind of protein in the protein material is of minor importance, it may be any vegetable protein or microbial protein, which is suitable for nutrition purposes. Preferably, the edible protein material of component A is an isolate. Such a protein isolate generally has an analytical protein content in the range of 80 to 95% in dry matter.

Examples of vegetable proteins are protein materials from pulses, such as chickpea, faba bean, lentils, lupine, mung bean, pea or soy, protein materials from oil seed, such as hemp, rapeseed/canola or sunflower, protein materials from cereals, such as rice, wheat or triticale, further potato protein, and protein materials from plant leaves such as alfalfa leaves, spinach leaves, sugar beet leaves or water lentil leaves, and algae protein and mixtures thereof.

Examples of microbial proteins, which are also termed single cell proteins (SCP) include fungal proteins, also termed mycoproteins, such as proteins from Fusarium venenatum, proteins from yeast such as proteins from Saccharomyces species, proteins from algae, such as proteins from spirulina or chlorella species, and bacterial proteins, such as proteins from lactobacilli species.

Preference is given to a protein component A, which comprises or consists to at least 90% by weight, based on the total amount of protein component A in the malleable mass, of one or more vegetable protein materials. In particular, the protein component A comprises or consists to at least 90% by weight, based on the total amount of protein component A in the malleable mass, of at least one vegetable protein material selected from isolates and concentrates of chickpea protein, faba bean protein, lentil protein, lupine protein, mung bean protein, pea protein or soy protein and mixtures thereof, with preference given to the isolates of the aforementioned protein material. In a particular group of embodiments, the component A comprises or consists to at least 90% by weight, based on the total amount of protein component A in the malleable mass, of at least one vegetable protein material selected from pea protein material and faba bean protein material or a mixture thereof, especially, if a fully allergen free product is required.

Vegetable protein materials as well as SCP having food grade are well known and commercially available.

Apart from water, the protein material is typically the main constituent of the malleable mass. It is generally constitutes at least 20% by weight and may constitute up to 75% by weight, based on the total amount of components different from water, hereinafter referred to as dry matter, in the malleable mass and calculated as the amount of protein material. As the protein material usually has an analytical protein content of at least 70% by weight, in particular of about 80 to 95% by weight in dry matter, the analytical protein content of the malleable mass is typically somewhat lower and constitutes frequently at least 16% by weight and up to 72% by weight, of the dry matter in the malleable mass. The amount of the component A is generally chosen such that the analytical protein content in the malleable mass is generally in the range of 5 to 18% by weight, in particular in the range of 7 to 16% by weight and especially in the range of 9 to 14% by weight. Usually, this corresponds to an amount of protein isolate in the range of 7 to 20% by weight, in particular in the range of 10 to 18% by weight and especially in the range of 13 to 16% by weight, based on the total weight of the malleable mass.

As a further component B, the malleable mass contains an organic polymeric gelling agent. According to the invention, the organic polymeric gelling agent is a water-soluble polysaccharide bearing carboxyl groups or are water soluble salts thereof, which are capable of being gelled by calcium ions. If the polysaccharide bearing carboxyl groups is not sufficiently water soluble, it is typically used as a water-soluble salt thereof. Water soluble salts include the alkali metal salts, in particular the sodium salts, and the ammonium salts, with preference given to the sodium salts.

Preferably, the polysaccharide bearing carboxyl groups is a polysaccharide wherein the majority of saccharide units, in particular at least 65 mol-% of the saccharide units, which form the polysaccharide, are uronic acid units, such as units of guluronic acid, mannuronic acid and galacturonic acid. The uronic acid units are preferably 1,4-connected. Examples of carboxyl groups bearing polysaccharides which are capable of being gelled with calcium ions are alginates and pectins.

Alginates are well known gelling additives in food. They are authorized food additives, namely E400 to E405. Amongst alginates, preference is given to sodium alginate. Likewise pectins are well known gelling additives in food (E440). Preference is given to low-methoxy pectins and their salts.

According to the invention, the concentration of the component B in the malleable mass is in the range of 1 to 3.3% by weight, in particular in the range of 1.1 to 2.8% by weight, especially in the range of 1.2 to 2.3% by weight, based on the total weight of the malleable mass. Preferably, the weight ratio of the total amount of the component A to the component B to in the malleable mass is in the range of 2:1 to 20:1.

Preferably, the component B is selected from the water-soluble salts of alginic acid, in particular the sodium salts, low-methoxy pectins and their water soluble salts and mixtures thereof.

In a very preferred group of embodiments, the component B is a water soluble salt of alginic acid, hereinafter referred to as alginate. The preferred alginate is sodium alginate. The amount of alginate in the malleable mass is in particular in the range of 1.1 to 2.8% by weight, especially in the range of 1.2 to 2.3% by weight, based on the total weight of the malleable mass and calculated as sodium alginate, also referred to as E 401.

In another group of embodiments, the alginate is partly or totally replaced by one or more other polysaccharide bearing carboxyl groups, which are capable of being gelled by calcium ions. Such polysaccharides that are different from alginate include but are not limited to pectins, in particular low-methoxy pectins and their water soluble salts These polysaccharide bearing carboxyl groups may be used in their acidic form or in the form of their alkali metal salts, and in particular in the form of their sodium salts. Preferably, the amount of such polysaccharide bearing carboxyl groups will not exceed the amount of alginate. In particular, the amount of alginate will typically make up at least 80% by weight of the total amount of alginate and other polysaccharide bearing carboxyl groups.

Especially, the alginate is the sole gelling agent B contained in the malleable mass.

The malleable mass may further contain a non-ionic polysaccharide, which is water-swellable, i.e. which forms a gel when it is dissolved or swollen in cold water (component C). As a non-ionic polysaccharide, particular preference is given to methyl cellulose, also referred to as E461. The non-ionic polysaccharide, in particular methyl cellulose, serves for modifying the hardness of the particles and particularly increases the thermal stability of the fibre. The presence of the non-ionic polysaccharide, in particular methyl cellulose, reduces the generally observed loss of hardness of the fibres when heated for hot consumption and thus better preserves the texture. If present, the amount of non-ionic polysaccharide, in particular of methyl cellulose, is generally in the range of 0.05 to 1% by weight, in particular 0.1 to 0.9% by weight, especially 0.2 to 0.8% by weight, based on the total weight of the malleable mass.

Preferably, the concentration of the non-ionic polysaccharide of component C in the malleable mass is chosen such that the mass ratio of component A to component C is in the range of 14:1 to 140:1 and the mass ratio of component B to component C is in the range of 1.5:1 to 20:1.

As pointed out above, a suitable ratio of protein component A, component B and component C is required to achieve a proper hydration of these components in the malleable mass. In this regard, it was found beneficial, if the total amount of component B and component C is in the range of 1.0 to 3.4% by weight, in particular in the range of 1.4 to 2.8% by weight, based on the total weight of the malleable mass.

In this regard, it was found particularly beneficial, if the mass percentage amounts of the components A, B and C is such that the following equation (I) is fulfilled:

X=a*A+b*B+c*C (I)
where [A], [B] and [C] are the mass percentages of components A, B and C, respectively,
where
a represents a number in the range of 2.5 to 5, in particular in the range of 3.5 to 4.5 b represents a number in the range of 10 to 25, in particular in the range of 15 to 20 and c represents a number in the range of 10 to 100, in particular in the range of 20 to 50, and where X represents a number in the range of 90 to 110.

Preferably, the concentrations of the respective components A, B and C in the malleable mass are chosen such that the mass ratio of component A to component B is in the range of 2:1 to 20:1, the mass ratio of component A to component C is in the range of 14:1 to 140:1 and the mass ratio of component B to component C is in the range of 1.5:1 to 20:1.

The malleable mass further contains an edible fat or oil, which are hereinafter also referred to as component D. Preferably, the component D is a vegetable fat or oil, in order to qualify the product as vegan. Apart from that, the type of fat or oil is of minor importance. Suitable vegetable fats or oils include, but are not limited to oils commonly used for cooking such as sunflower oil, corn oil, rapeseed oil, including also canola oil, coconut oil, cottonseed oil, olive oil, peanut oil, palm oil, palm kernel oil, safflower oil, soybean oil, sesame oil, and mixtures thereof. The edible fats or oils may also include nut oils, oils from stone fruits such as almond oils and apricot oil, oils form melon or pumpkin, flaxseed oil, grapeseed oil, and the like and mixtures thereof with the aforementioned fat or oils for cooking. In particular, the amount of fats or oils used commonly used for cooking amount to at least 50% by weight, based on the total amount of fat or oil in the malleable mass. The amount of oil in the malleable mass may vary and may be as low as 1% by weight or as high as 15% by weight. preferably, the total amount of edible fat or oil in the malleable mass is in the range of 3 to 12% by weight, especially in the range of 5 to 10% by weight, based on the total weight of the malleable mass.

Apart from that, the malleable mass contains water as component E. The amount of water is generally in the range of 60 to 90% by weight, in particular in the range of 65 to 85% by weight or 69 to 80% by weight or 73 to 78% by weight, based on the total weight of the malleable mass, of water.

In particular, the malleable mass contains
a) 7 to 20% by weight, in particular 8.5 to 18% by weight or 10 to 18% by weight and especially 13 to 16% by weight based on the total weight of the malleable mass, of the protein component which typically corresponds to an analytical protein content in the malleable mass in the range of 5 to 18% by weight, in particular in the range of 7 to 16% by weight and especially in the range of 9 to 14% by weight;
b) 1 to 3.3% by weight, in particular 1.1 to 2.8% by weight, especially 1.2 to 2.3% by weight, based on the total weight of the malleable mass, of component B, where the component B is in particular alginate or a mixture thereof with a pectin, and where the component B is especially sodium alginate;
c) optionally 0.05 to 1% by weight, in particular 0.1 to 0.9% by weight, especially 0.2 to 0.8% by weight, based on the total weight of the malleable mass, of the nonionic polysaccharide, in particular methyl cellulose;
d) 1 to 15% by weight, in particular 3 to 12% by weight, especially 5 to 10% by weight, based on the total weight of the malleable mass, of the component D, i.e. an edible fat or oil of plant origin; and
e) 60 to 90% by weight, in particular 65 to 85% by weight or 69 to 80% by weight or 73 to 78% by weight, based on the total weight of the malleable mass, of water.

A skilled person will immediately understand that the total amount of the ingredients of the malleable mass will add to 100% by weight and any combination of the aforementioned amounts that deviates from 100% by weight will be compensated by reducing or increasing the amount of water.

Furthermore, the malleable mass may contain small amounts of starch flour or plant fibres such as citrus fibre. The total amount of such ingredients will generally not exceed 1% by weight of the malleable mass and may be in the range of 0.01 to 1% by weight, based on the total weight of the malleable mass.

Furthermore, the malleable mass may contain small amounts of additives conventionally used in edible protein materials, which include, but are not limited to, sweeteners, spices, preservatives, color additives, colorants, antioxidants, etc. The total amount of such ingredients will generally not exceed 1% by weight of the malleable mass and may be in the range of 0.01 to 1% by weight, based on the total weight of the malleable mass.

In step (i) the malleable mass is generally prepared by mixing the ingredients of the malleable mass in their respective amounts, preferably with shearing. Usually, the components A, B and C are added to the water in an arbitrary order or as a pre-blend in a suitable mixing device, followed by the addition of oil. If the malleable mass contains the component C, especially methyl cellulose, it may be added together with the components A and B. Although component C is a powder and thus can be added as such, it is beneficial, if it is used as a solution in water, e.g. as a 0.1 to 5% by weight aqueous solution. In particular, component C, especially methyl cellulose is used in its pre-hydrated form. For this, component C, especially methyl cellulose, is mixed with cold water, which preferably has a temperature in the range of 0 to <20° C., in particular 0 to <10° C., with shearing to obtain a virtually homogeneous gel of hydrated methyl cellulose. For obtaining the pre-hydrated component C typically about 1 to 5 g of component C per 100 g of water are used.

Preferably, the components of the malleable mass are mixed with shearing. Mixing and shearing can be carried out successively or simultaneously. Shearing results in a homogenization of the component in water such that they are evenly distributed. Suitable apparatus for mixing and shearing include bowl choppers, cutters, such as Stephan cutters, high speed emulsifiers, in particular those based on the rotor-stator principle, colloid mills and combinations thereof with a blender. The thus obtained malleable mass has typically a dough like consistency.

The malleable mass is generally prepared at temperatures in the range of 10° C. to 95° C., in particular in the range of 72° C. to 90° C. In other words, mixing and optional shearing is carried out at these temperature ranges.

In step (ii) of the process of the invention, the malleable mass is comminuted. Thereby, the malleable mass is comminuted into particles, which are mechanically instable. By bringing the particles in contact with the aqueous solution of the calcium salt in step (iii), the calcium ions will immediately crosslink the alginate molecules and thus also gellify/precipitate the particles on the particle's surface. Thereby, a rigid skin on the surface of the particles is formed, which stabilize the particles. Upon prolonged contact of the particles with the aqueous solution of the calcium salt in steps (iii), the calcium ions will diffuse into the interior of the particles and gellify/precipitate the component A and the component B in the interior of the particles, resulting in a hardening of the particles.

Comminution of the malleable mass (i.e. step (ii)) and bringing thus formed particles into contact with the aqueous solution of the calcium salt (iii) can be carried out simultaneously or successively. Step (iii) may be divided in an initial step (iii.a), which is carried out immediately after step (ii) or simultaneously with step (ii) and a final step (iii.b). In step (iii.a) the mechanically instable particles obtained by comminution are stabilized due to the formation of a rigid skin while in step (iii.b) the particles are allowed to rest in a solution of the calcium salt until they have achieved their final hardness. The total time for achieving the final hardness will typically be in the range of 6 h to 24 h, in particular in the range of 8 h to 20 h.

The hardening of step (iii) is generally carried out at temperature in the range of 0 to 95° C., in particular either in the range of 0 to 20° C. or at a temperature of at least 50° C., e.g. in the range of 50 to 95° C. and in particular in the range of 50 to 75° C. Therefore, phase (iii.b) is also preferably carried out at a temperature of at least 50° C., e.g. in the range of 50 to 95° C. and in particular in the range of 50 to 75° C. Higher temperatures during the contact of the solution with the particles formed from the malleable mass favor the diffusion of calcium ions into the particles and thus reduce the hardening time.

Regardless of whether comminution of the malleable mass and bringing thus formed particles into contact with the aqueous solution of the calcium salt is carried out simultaneously or successively, the aqueous solution of the calcium salt has generally a concentration of calcium in the range of 0.5 to 1.5% by weight, based on the total weight of the aqueous solution of the calcium salt and calculated as elemental calcium. Higher concentrations of calcium salt will favor the diffusion of calcium ions into the particles formed from the malleable mass and thus reduce the hardening time. The type of calcium salt for producing the aqueous solution is of minor importance, as long as it is sufficiently soluble in water at the respective temperature and is acceptable for nutritional purposes. Suitable salts for producing the solution, which are sufficiently soluble, include, but are not limited to calcium chloride, calcium lactate, calcium gluconate. The pH of the aqueous solution is of minor importance, preferably the aqueous solution of the calcium salt has a pH in the range of about pH 4 to about pH 8 as determined at 20° C.

The temperature of the aqueous solution of the calcium salt is typically in the range of 0 to 95° C., in particular in the range of 50 to 75° C. Preferably, the temperature of the aqueous solution of the calcium salt is such that during the mixing/comminution/curing, a temperature in the range either of 0 to 20° C. or at least 50° C., e.g. in the range of 50 to 75° C. is maintained.

Regardless of whether comminution of the malleable mass and bringing thus formed particles into contact with the aqueous solution of the calcium salt is carried out simultaneously or successively, the mass ratio of the aqueous solution of the calcium salt to the particles formed from the malleable mass is in the range of 1:3 to 3:1, in particular in the range of 1:2 to 2:1 and especially of about 1:1.

For an aforementioned mass ratio of the aqueous solution of the calcium salt to the particles of 1:1, preferably, the ratio of the percentage of calcium ions in the solution to the percentage of component B in the particle is in the range of 0.25:1 to 1:1, but should not be lower than 0.2:1. The percentage of calcium ions in the aqueous should be adjusted, if another mass ratio of aqueous solution to the particles is applied; e.g. for a mass ratio of 1:3, the lower limit of the percentage of calcium ions in the aqueous should be preferably at least 0.6:1, in particular at least 0.75:1. For a mass ratio of higher than 1:1 the ratio of the percentage of calcium ions in the solution to the percentage of component B in the particle may be lower than 0.25:1.

Generally, the comminution of the malleable mass is carried out such that the majority of the formed particles, i.e. at least 90% by weight of the particles, are not too small but also not too big and have a size of at least 5 mm, e.g. in the range of 5 to 100 mm, and in particular, in its smallest spatial distance, in the range of 10 to 50 mm.

As explained above, a rigid skin is formed on the surface of the particles formed by comminution, while the particles are in contact with the aqueous solution of the calcium salt. The formation of the rigid skin occurs quite rapidly and generally contact times of e.g. at least 1 minute in particular at least 2 minutes are necessary to obtain a sufficient stability for handling the particles. This time period is also referred to as step (iii.a). Therefore, it may be possible to remove the particles from the solution of the calcium salt after a short while and to transfer them into a second aqueous solution of the calcium salt, where they are allowed to rest until they have achieved their final hardness. This step is also referred to as step (iii.b). For practical reasons contact times in this initial phase (iii.a) may be in the range of 2 to 60 minutes, in particular in the range of 2 to 30 minutes, especially in the range of 2 to 15 minutes are preferred. During this phase (iii.a), a temperature of preferably either in the range of 0 to 20° C. or at least 50° C., e.g. in the range of 50 to 75° C., is maintained.

After the initial contact time, the particles can be separated from the aqueous solution of the calcium salt and the particles are transferred into a second aqueous solution of a calcium salt, wherein the particles will rest to achieve their final hardness (phase (iii.b)). Separation of the aqueous solution of calcium salt can be achieved by conventional methods of separating coarse solids from liquids, e.g. by sieving the mixture of particles and the aqueous solution of calcium salt or by decantation of the aqueous solution from the particles. For example, the mixture of particles and the aqueous solution of the calcium salt can be rinsed through a sieve or the particles can be removed from the solution with a sieve plate or by transporting the preformed particles (floating and swimming in the solution) with a belt conveyor, e.g. an inclined haulage conveyor, from the precipitation solution into the second aqueous solution of the calcium salt, where the particles are allowed to harden. Thereby, the particles achieve their final hardness (phase (iii.b)) which is generally after a total contact time of the particles with the solution of the calcium salt in the range of 6 to 24 h, in particular in the range of 8 to 20 h. Phase (iii.b) may be carried out at temperatures in the range of 0 to 95° C., in particular at a temperature of either in the range of 0 to 20° C. or of at least 50° C., e.g. in the range of 50 to 75° C. with preference given to the latter.

If comminution of the malleable mass and bringing the thus formed particles into contact with the aqueous solution of the calcium salt is carried out simultaneously, the malleable mass is comminuted in the presence of the aqueous solution of the calcium salt. In this case, comminution is typically carried out by stirring or kneading the mixture of the malleable mass and the aqueous solution of the calcium salt. For example, the total aqueous solution of the calcium salt may be added to the malleable mass, while comminuting the mass into particles, e.g. by stirring or kneading, e.g. in a paddle mixer over a period of time, e.g. for 5 to 15 min. For this, the aqueous solution may be added to the malleable mass or the malleable mass is added to the aqueous solution of the calcium salt and the comminution in the thus obtained mixture. Comminution is carried out such that the majority of the formed particles, i.e. at least 90% by weight of the particles, are not too small and have a size in the ranges given above. The thus obtained particles may rest in the solution of the calcium salt until they have achieved their final hardness. It is also possible to remove the mixture of particles with the solution from the mixer, when they have a sufficient stability for further handling, and transfer them together into a second container, where they are allowed to rest or are gently mixed until they have achieved their final hardness. If only the particles are separated from the first vessel, they have to be put into the second container with a fresh aqueous solution of the calcium salt in a balanced concentration and ratio to the emulsion as described above. It is also possible to continuously add the malleable mass to the solution of the calcium salt with comminution of the mass into particles and continuously remove the particles from the solution, when they have a sufficient stability for further handling, and to transfer them into a second container with a solution of the calcium salt, where they are allowed to rest or are gently mixed until they have achieved their final hardness.

Preferably, comminution of the malleable mass and bringing the thus formed particles into contact with the aqueous solution of the calcium salt are carried out successively. For this, step (ii) preferably comprises passing the malleable mass through a grid or a perforated plate into the aqueous solution of the calcium salt. It is also possible to pre-shape the mass by combined filling and cutting device, e.g. by a ball former with a diaphragm knife system. By passing the malleable mass through a grid, a perforated plate or a diaphragm, particles are formed, which have a size essentially defined by the size of the perforation of the plate or the mesh size of the grid or the diaphragm, respectively. The thus formed particles are then introduced into the aqueous solution of the calcium salt. Preferably, the aqueous solution is stirred while the particles of the malleable mass are introduced into the solution, in particular, if the initially formed particles need to be further comminuted. Thus the particle size can also be adjusted by the intensity of the stirring. The thus obtained particles must rest in the solution of the calcium salt until they have achieved their final hardness. It is also possible to remove the particles from the solution, when they have a sufficient stability for further handling and transfer them into a second solution of the calcium salt, where they are allowed to rest until they have achieved their final hardness. It is also possible to continuously comminute the particles and introduce them into the solution and continuously remove the particles from the solution, when they have a sufficient stability for further handling, and to transfer them into a second solution of the calcium salt, where they are allowed to rest until they have achieved their final hardness.

After the particles have achieved the desired final hardness, they are removed from the aqueous solution of the calcium salt. For example, the mixture of particles and the aqueous solution of the calcium salt can be rinsed through a sieve or the particles can be removed from the solution with a sieve plate. Separation can be operated bath-wise or in continuous mode.

The hardened particles can be optionally heat treated to a core temperature of ≥72° C. for better shelf-stability and are cooled to and stored cool at temperatures of ≤5° C., e.g. in a refrigerator, or are deep frozen and kept at temperatures of below −18° C. in a deep freezer.

The particles obtainable by the process of the invention are particularly suitable for producing meat substitute products. For this, the particles are processed to meat substitute products by analogy to known methods as described in the prior art. For example, the meat substitute products can be produced by mixing the particles with binders of non-animal origin, such as hydrocolloids or plant fibres, and/or with herbs and spices, followed by shaping them to the desired shapes e.g. by using moulds or casings. The thus obtained shaped products can be portioned, optionally coated, e.g. with batters, breadcrumbs or external seasonings. Then the products are chilled, frozen or pasteurized and packaged for distribution as finished meat substitute products such as burgers, nuggets, fish fingers, schnitzels, sausages and the like.

The invention is hereinafter explained by the following experiments, describing the characteristic properties of the fibre and fibre process and by the related figures.
A) Hardening rate and Final Hardness:
   1) Testing of influencing parameters
   2) Development of the hardening over Processing time
   3) Influence of curing temperature on hardening process
B) Relations of overall dry matter, protein, alginate and methyl cellulose contents on fibre producibility and hardness
   4) Setting of alginate by calcium diffusion into the emulsion
C) Reduction of Alginate—influence of different compositions of protein, alginate and methyl cellulose on final hardness and hardening time
   5) Varied proportions of protein to alginate
   6) Influence of methyl cellulose
   7) Firmness of fibres depending on the curing time
   8) Mimicking typical hot consumption temperature of finished meat substitute products

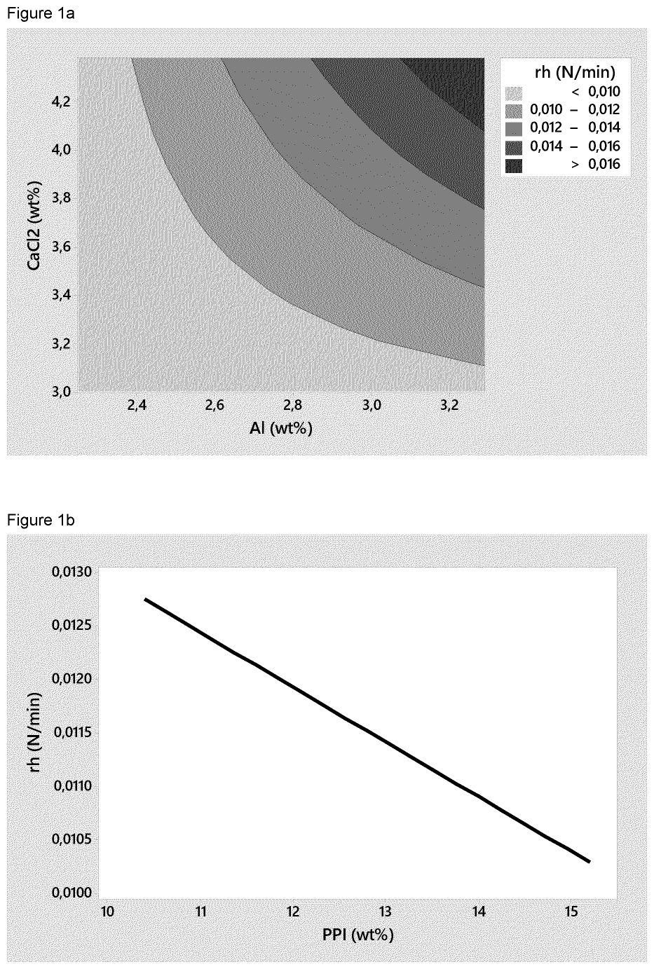

FIG. 1: a) Influence of Alginate fraction in emulsion and Calcium Chloride-dihydrate fraction in the curing solution on the hardening rate;

b) Influence of the PPI-concentration (in the emulsion) on the hardening rate.

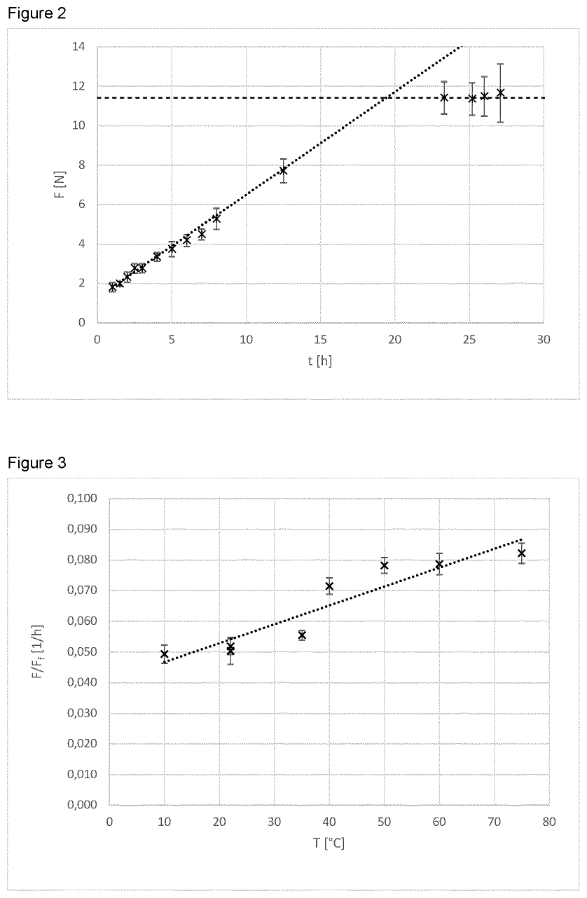

FIG. 2: Force development of the reference experiment.

FIG. 3: Impact of temperature on hardening.

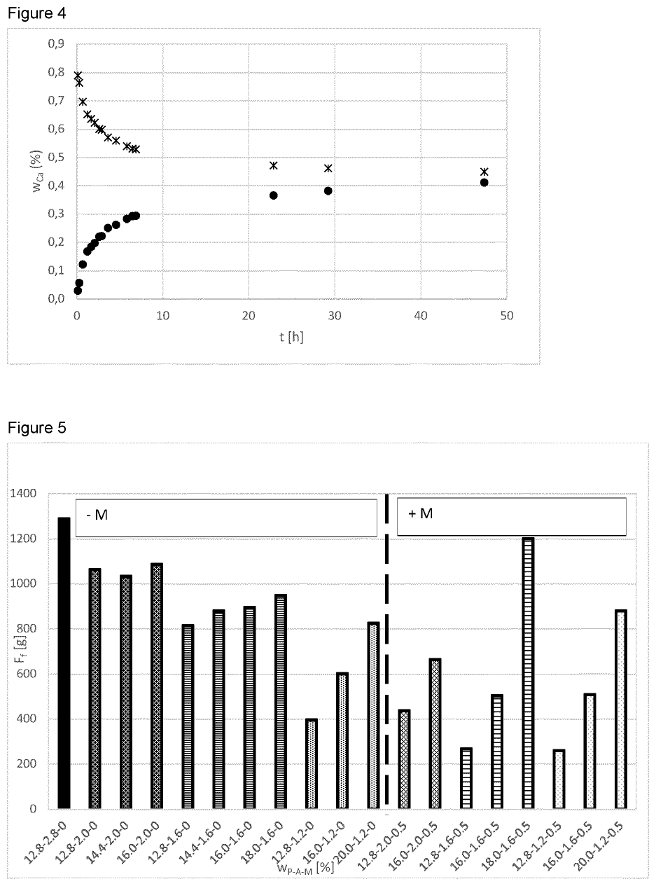

FIG. 4: Distribution of mass fraction of calcium in fibre and precipitation solution during hardening.

FIG. 5: Total Hardness of alginate-reduced/protein-increased fibres without and with methyl cellulose.

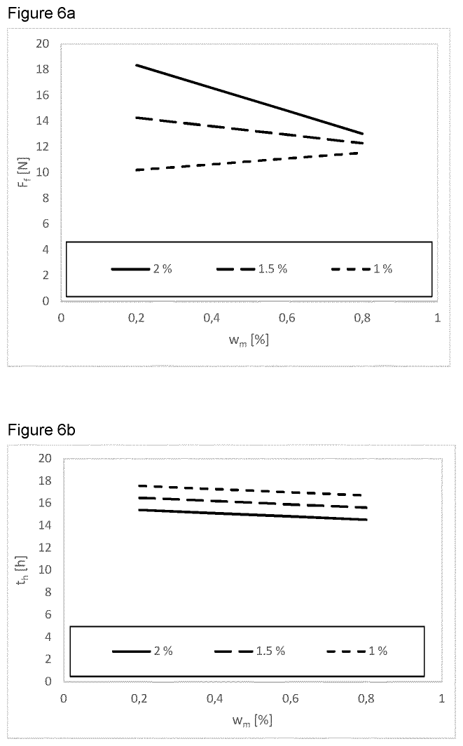

FIG. 6: a) Final Hardness in dependence of alginate and protein-content, without or with methyl cellulose, at 14% PPI.

b) Hardening time in dependence of alginate and protein-content, without or with methyl cellulose, at 14% PPI.

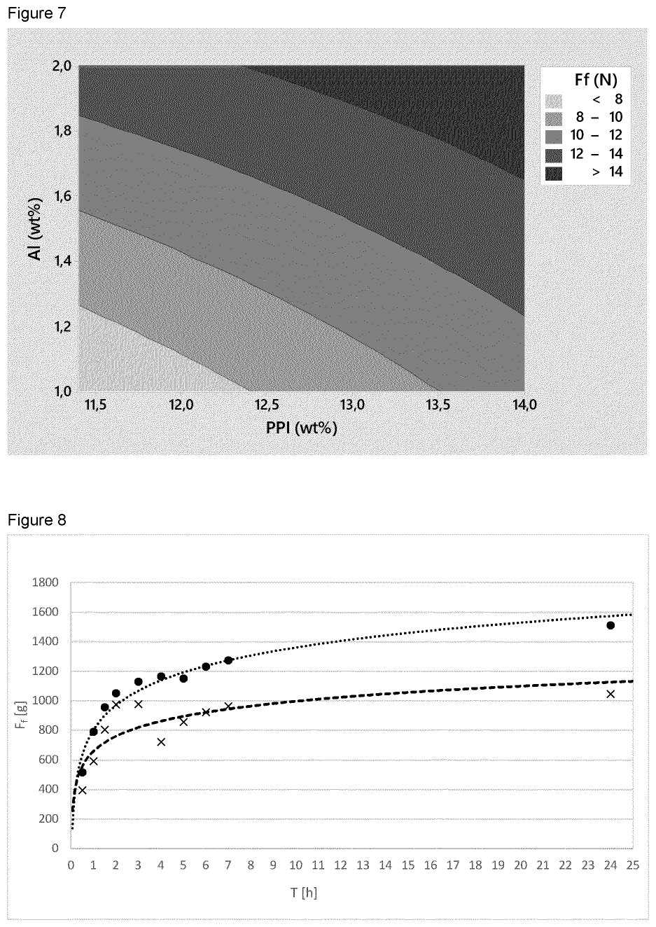

FIG. 7: Correlation of alginate and PPI on the final hardness.

FIG. 8: Firmness of fibres depending on the curing time in the CaCl₂-solution at room temperature.

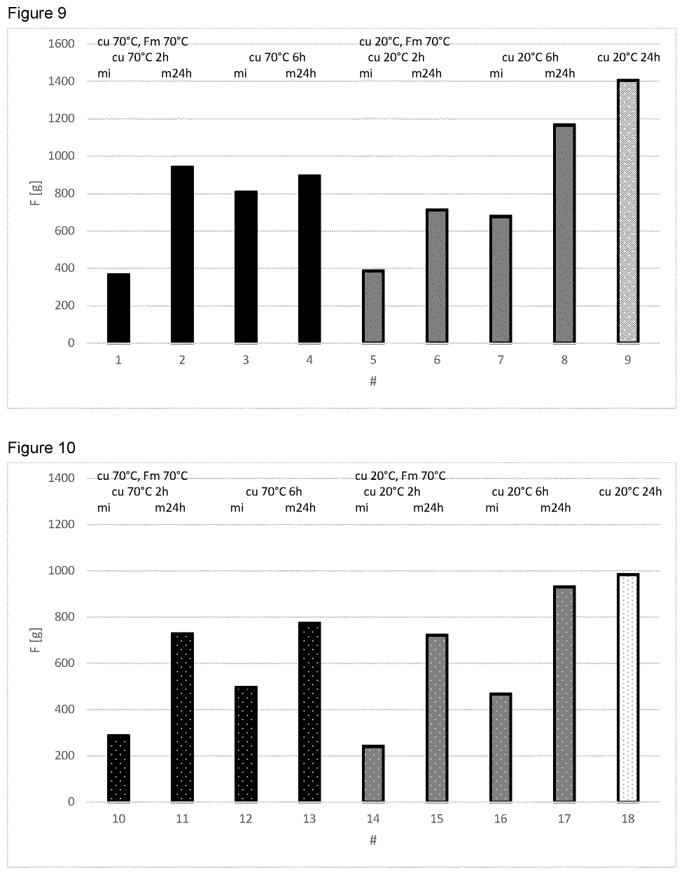

FIG. 9: Fibres with higher alginate content, hardened at 20 or 70° C., but firmness measured at 70° C.

FIG. 10: Fibres with lower alginate content plus methyl cellulose, hardened at 20 or 70° C., but firmness measured at 70° C.

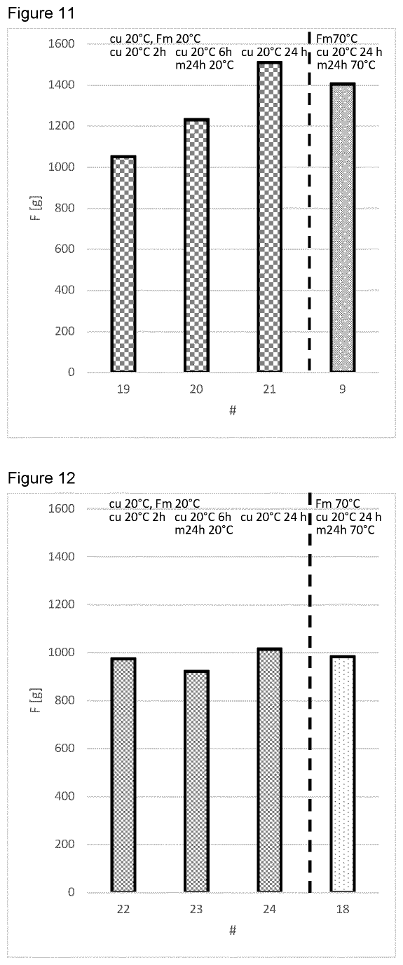

FIG. 11: Fibres with higher alginate content, hardened at 20° C., firmness measured at 20 and 70° C.

FIG. 12: Fibres with lower alginate content plus methyl cellulose, hardened at 20° C., firmness measured at 20 and 70° C.

In the examples, the following abbreviations are used:
MC: methyl cellulose
MCg: methycellulose gel
pbw parts by weight
PPI: pea protein isolate
rpm: revolution per minute
CaCl₂ calciumchlorid-dihydrate (all mass fractions given for CaCl₂ are related to the dihydrate, if not otherwise mentioned)
wt % % by weight
SO sunflower oil
Na-A sodium alginate
DoE Design of experiment

Here and in the following the terms “emulsion” and “malleable mass” are used synonymously.

Here and in the following the terms “particle” and “fibre” are used synonymously.

### (Standardized) Preparation and Measuring Method of Hardness

The following ingredients were used:
Pea protein isolate having a protein content of approx. 85% by weight in dry matter, obtained from Cosucra Groupe Warcoing—Pisane M9 or AGT Foods—Pea Protein 85
Sodium alginate with purity of >90.8% calculated as sodium alginate, e.g. commercial product of Hewico—Hewigum NA 1
Calciumchloride-dihydrate Merck KgaA—Calcium Chloride Dihydrate cryst.
Methyl cellulose, J. Rettenmaier & Söhne GmbH—Vivapur Methyl Cellulose MC A4M

pH values were determined by a pHenomenal 1100 L by VWR using a glass electrode.

Force measurements: Final hardness and hardening time. Compression force was measured with an Imida FCA-DSV-50N-1 (F expressed in N) or a TATexturizer (F expressed in g) with a 20 mm cylindrical stamp.

Conductivity was measured by using an Ahlborn Almemo® 710 measuring instrument in combination with the D7 conductivity sensor FYD 741 LFE01.

Calcium was measured from the ash by IC (ion chromatography) with a ThermoFisher Scientific/Dionex ICS-1000 Ion Chromatography System.
1) General protocol of determining the hardening time and final hardness of hardened protein mass with diffusion setting:
1.1 For the following tests varied recipes of a protein mass, hereinafter referred to as protein emulsion or emulsion or as malleable mass, were used. The emulsion is prepared by mixing indicated percentages of pea protein isolate, alginate, with 9 parts by weight of a vegetable oil, e.g. sunflower oil (if not otherwise mentioned) or rapeseed oil or canola oil and water to obtain a protein emulsion. The amount of water was adjusted to obtain 100 parts by weight of the emulsion. Mixing was carried out in a Thermomix TM5 at >70° C.-90° C. for about 3 min.
1.2 For curing, 10 g of the emulsion was placed into a cylindrical tube with 33 mm diameter and covered with 10 g of a 2-5% by weight (percentage as indicated) aqueous solution of CaCl₂-dihydrate. The emulsion mass was scratched from the cylinder wall, thus allowing the emulsion to be undercut by the solution until a sphere is formed which is cured in the solution for the indicated time (in general up to 24 hours) at a defined temperature, either at 20 or 72° C., as indicated. After the curing by calcium diffusion into the spherical fibres, they are taken out of the solution and allowed to drip. Resulting particles have a diameter of approx. 25 mm. A similar form is required for the force measurements made during the curing process, otherwise the results cannot be compared.
1.3 Then firmness/hardness is assessed by a texture analysis measurement at the selected temperature using the following conditions: 3 spherical particles per experiment, measured 3 to 5 times each, compressed 5 mm.
1.4 The hardness measured after 24 h is assumed to be the final one. For the calculation of the hardening time the development of the hardness over time is evaluated. Between the data points of the first 4 h a linear regression is performed. The time at which the regression reaches the final hardness is called the hardening time.
1.5 In order to get comparable hardening rates for samples with a different final hardness, the relative hardening rate is introduced. The hardness of each measurement is divided by the final one, accordingly the plot ends at the border line of final hardness which corresponds to a reference value of 1 (100%).

### A) Hardening Rate and Final Hardness

### 1: Design of Experiments for Testing of Influencing Parameters

After an initial test with more variables and a wider range for each parameter, a design of experiments was carried out with 17 test groups in a high level of detail for the most significant three parameters, for which ingredient ratios were limited to smaller ranges: the PPI fraction was ranging from 10.4-15.2 wt % and alginate from 2.25 to 3.29 wt % in the emulsion, 9 wt % vegetable oil (sunflower) was kept constant and water as a balance to 100 wt % adjusted. The concentration of calcium chloride-dihydrate in the aqueous solution used for precipitation/hardening (hereinafter precipitation fluid) was ranging from 3 to 4.38 wt %. Less-significant parameters were fixed on pH≈7, mixing temperature of 90° C. and emulsion mixing time to 3 min.

Water and oil were provided into the Thermomix and dispersed. Afterwards, PPI and alginate were added, the mass was heated up to 90° C. and stirred at stage 3-4 for 3 min. until the mixture was homogeneous.

The thus obtained emulsions were subjected to a diffusion hardening for 24 h at 20° C. according to the protocol described under 1.2. The development of the hardening rate was assessed by measuring the compression force periodically according to the protocol of example 1.3. Final hardness was determined according to 1.4 above.

FIGS. 1a and 1b show the interaction of alginate and calcium salt, exemplarily shown for 14% PPI) and the smaller effect of PPI at different concentrations on the hardening rate.

FIG. 1a shows the influence of the alginate fraction in the emulsion and calcium fraction in the precipitation fluid, given as calcium chloride-dihydrate fraction, on the hardening rate rh given in the contour lines as N/min. FIG. 1a is a contour-plot-graph of hardening rate rh [N/min], where the x-axis is the alginate fraction Al in wt % and the y-axis is the concentration of CaCl₂-dihydrate in the precipitation fluid in wt %.

FIG. 1b shows the influence of the PPI-concentration in the emulsion on the hardening rate. FIG. 1b is a one factor analysis of a contour-plot-graph of hardening rate rh [N/min], where the x-axis is the PPI fraction in wt % and the y-axis is hardening rate [N/min].

As can be concluded from FIGS. 1a and 1b, the main factors affecting the hardening rate are the calcium fraction in the hardening solution and the alginate fraction in the emulsion and their interaction with each other. As visible from this trial more calcium and more alginate result in a faster hardening. A higher fraction of PPI in the emulsion results by trend in a slight decrease of the hardening rate. Probably the addition of solid in the form of protein hinders the diffusion of calcium into the samples.

### Experiment 2: Development of the Hardening Over Processing Time

A reference experiment with following compositions was carried out to demonstrate the compression force/the development of the hardening over the course of time at 20° C. up to a day according to protocols described under 1.1 to 1.5:Emulsion: 78.4 wt % water, 9 wt % sunflower oil, 10.4 wt % PPI, 2.2 wt % alginate.

Precipitation Fluid: 3 wt % calcium chloride dihydrate in water.

FIG. 2 shows the force development of the reference experiment. Additionally, the linear regression from the first 4 h is plotted. In FIG. 2, the following abbreviations are used:
F [N]=Compression Force
t (h)=time in hours
x=measured hardness
. . . . . =Regression first 4 h
- - - - - - =Final hardness (24 h)

A linear regression during the first 4 h represents the following 8 h well, too. The final hardness is almost constant after finishing the process. The intersection of the diagonal with the greatest hardness is the time for complete hardening.

### Experiment 3: Influence of Curing Temperature on Hardening Process

In a third experiment, the additional influence of the process temperature on the hardening process was measured for the same composition as in experiment 2. A Genie Temp-Shaker 300 was used to set fixed temperatures during the hardening procedure according to protocol 1.2. To avoid temperature gradients a shaking rate of 80 rpm was applied every time. The development of the hardening was assessed by measuring the compression force according to the protocol 1.3. Final hardness was determined according to protocol 1.4 and relative hardening rate was calculated according to protocol 1.5.

In order to achieve a faster hardening rate and achieving the desired final hardness of the fibre, the temperature of the aqueous solution of the calcium salt should preferably be below the emulsification temperature of 70-90° C. Nevertheless, it should preferably remain in a relatively high temperature range, preferably >50° C., or rather >60° C. or—taking into account shelf-stability reasons—even at a temperature ≥72° C. A significant increase of relative hardening rate and accordingly reduction of processing time was observed as can be seen from FIG. 3. Therefore, a temperature in the range of 50 to 72° C. would also reduce the pure process time.

FIG. 3 shows the dependence of the relative hardening rate F/F_{f} from the temperature. The relative hardening rate refers to the quotient of hardness of each measurement (F) divided by final hardness (F_{f}) and is given in 1/h. In FIG. 3 T [° C.] refers to the temperature in ° C. during hardening. From the measured data, the following equation for the dependency of the relative hardening rate from the temperature was established by linear regression:

Hardening rate=hardness of each measurement (F) divided by Final Hardness (F_{f}=F/F_{f}[1/h])

F/F_{f}[1/h]=6,15E-04*T[° C.]+4,05E-02.

Whilst with increasing temperature, the final hardness decreases slightly, which is probably due to different alginate gel network properties at higher temperatures, a clear trend can be seen considering the relative hardening rate. It increases almost linear with increasing temperature due to the increased diffusion coefficient of calcium.

Significant reduction of the processing time is guaranteed at higher process temperatures; increasing the process temperature from room temperature to 75° C. results in an increase of the relative hardening rate of 70%, equivalent to a reduction of the processing time by 40%.

### B) Relations of Overall Dry Matter, Protein, Alginate and Methyl Cellulose Contents on Fibre Producibility and Hardness

Experiment 4: Setting of Alginate by Calcium Diffusion into the Emulsion

Hardened particles were produced based on the same composition as in protocol 1.1-78.4 wt % water, 9 wt % sunflower oil, 10.4 wt % PPI, 2.2 wt % alginate—and cured in an aqueous 3 wt % calcium chloride-dihydrate-solution in several vessels until full hardening has been achieved. During hardening, the concentration of the calcium in the hardening fluid was monitored by measuring the conductivity in the calcium chloride solution, the bulk phase; additionally calcium concentrations were analyzed in solution and particles by IC from one vessel at any time.

In FIG. 4 both the calcium concentration * in wt % calcium in the precipitation fluid and calcium concentration ● in wt % calcium in the particles are plotted vs. time.

Concentrations of the calcium fraction in the bulk phase (precipitation fluid) and the particles during the hardening process are shown.

The FIG. 4 shows the quantitative shift (diffusion) of the calcium from the solution into the precipitated fibre during the hardening process. As soon as alginate is in contact with calcium, a gelation occurs forming a hard skin around the fibres, leading to a whole gelation of the fibre with further diffusion of calcium from the curing solution into the core. In FIG. 4 the following abbreviations are used:
w_{ca}(%)=mass fraction of calcium [wt %], calculated as elemental calcium
t (h)=time in hours
*=Precipitation solution
●=Fibre

Due to the diffusion of calcium from the precipitation solution into the particles the concentration in the solution decreases while the concentration of calcium in the particles increases. When the mass fractions are the same, the process is in principle finished. In practice, depending on the hardening time, it can be even more than half of the calcium that migrates into the particles, as the calcium bound to the alginate disappears out of the balance.

### C) Reduction of Alginate—Influence of Different Compositions of Protein, Alginate and Methyl Cellulose on Final Hardness and Hardening Time

In the following experiments, the proportions of protein to alginate and methyl cellulose, calcium chloride as a precipitant, and the type and time of addition of methyl cellulose were investigated. A possible compensation of a reduced amount of alginate was tested in comparison of final hardness to the reference process of protocol 1.

Methyl cellulose was hydrated under shearing in water at low temperatures (5° C.) and then added to the main emulsion and then emulsified with all other components. Additionally to the concentration of all components in some experiments other parameters like temperatures in different process steps and process time were varied.

Experiments 5: Total Hardness of Alginate-Reduced/Protein-Increased Fibres without and with Methyl Cellulose

In one series of tests 5.1-5.11 protein emulsions were prepared and hardened by analogy to the protocol 1, where the alginate fraction in the emulsion was gradually reduced from about 2.8 to 1.2% whilst PPI concentrations were gradually increased from about 12.8 to 20%. In a parallel test series 5.12-5.19 a 2% methyl cellulose gel in water (pre-sheared at low temperature, hereinafter 2% MCg) was incorporated into the base emulsion in an amount of 0.5 wt % MC with respect to the total malleable mass in order to evaluate its effect on compensation of lower alginate contents on hardness (see FIG. 5). The test layout followed the protocol 1.1-1.5, with a curing at 20° C.

This experiment demonstrates the producibility of typical protein fibres with higher protein contents, as requested by the market, with simultaneously reduced alginate contents, as regionally restricted by legal regulations, without disturbing the balance of hydration and processability, e.g. not risking a too dry, non-cohesive product or an excessively viscous, unmanageable mass during processing. At the same time, it was tested whether a reduced gel strength caused by reduced amounts of alginate can be compensated to a measurable extent by adding methyl cellulose.

The experimental setup is given in the following tables 1 and 2:

TABLE 1
Experiments without methyl cellulose

| Exp. 5. # | 1 | 2 | 3 | 4 | 5 | 6 | 7 | 8 | 9 | 10 | 11 |
|---|---|---|---|---|---|---|---|---|---|---|---|
| PPI [wt %] | 12.83 | 12.83 | 14.44 | 16.04 | 12.83 | 14.40 | 16.00 | 18.00 | 12.83 | 16.00 | 20.00 |
| Na-A [wt %] | 2.77 | 2.00 | 2.00 | 2.00 | 1.60 | 1.60 | 1.60 | 1.60 | 1.20 | 1.20 | 1.20 |
| Water [wt %] | 75.40 | 76.17 | 74.56 | 72.96 | 76.57 | 75.00 | 73.40 | 71.40 | 77.00 | 73.80 | 69.80 |
| SO [wt %] | 9.00 | 9.00 | 9.00 | 9.00 | 9.00 | 9.00 | 9.00 | 9.00 | 9.00 | 9.00 | 9.00 |
| MCg [wt %] | 0 | 0 | 0 | 0 | 0 | 0 | 0 | 0 | 0 | 0 | 0 |
| Total [wt %] | 100 | 100 | 100 | 100 | 100 | 100 | 100 | 100 | 100 | 100 | 100 |
| Ff [g] | 1288 | 1063 | 1033 | 1086 | 814 | 879 | 895 | 948 | 396 | 601 | 825 |

TABLE 2
Experiments with methyl cellulose

| Exp. 5. # | 12 | 13 | 14 | 15 | 16 | 17 | 18 | 19 |
|---|---|---|---|---|---|---|---|---|
| PPI [wt %] | 12.83 | 16.04 | 12.83 | 16.00 | 18.00 | 12.83 | 16.00 | 20.00 |
| Na-A [wt %] | 2.00 | 2.00 | 1.60 | 1.60 | 1.60 | 1.20 | 1.20 | 1.20 |
| Water [wt %] | 51.17 | 47.96 | 51.57 | 48.40 | 46.40 | 51.97 | 48.80 | 44.80 |
| SO [wt %] | 9.00 | 9.00 | 9.00 | 9.00 | 9.00 | 9.00 | 9.00 | 9.00 |
| MCg [wt %] | 25.00 | 25.00 | 25.00 | 25.00 | 25.00 | 25.00 | 25.00 | 25.00 |
| Total [wt %] | 100 | 100 | 100 | 100 | 100 | 100 | 100 | 100 |
| Ff [g] | 437 | 664 | 268 | 503 | 1200 | 260 | 508 | 880 |

FIG. 5 shows the total hardness F_{f} in g of alginate-reduced/protein-increased fibres without methyl cellulose (a) and with methyl cellulose (b)

The following abbreviations were used in FIG. 5:
F_{f}[g]=Final hardness in [g] (TA-measurement)
W_{P-A-M} [%]=concentrations of protein/alginate/methyl cellulose in %
−M=without methyl cellulose
+M=with methyl cellulose

Measurements of the hardness of particles of the experiments show that total hardness decreased with reduced alginate contents but increased again to a certain extent with considerably increased protein contents by which similar consistencies can be achieved. Thus with reduced alginate contents it is also possible to incorporate more protein without running into problems of insufficient hydration of the total emulsion.

When a pre-stabilized methyl cellulose gel is combined with these ratios firmness increases especially at lower alginate contents.

This implies that reduced amounts of alginate, down to a certain lower limit, either alone or in combination with methyl cellulose, are sufficient to gellify a mass containing protein, fat and water even with extended concentrations of protein and to achieve sufficient final strength of the fibre.

It was additionally observed that a water exchange takes place between all components, i.e. especially between the pre-stabilized methyl cellulose gel on one side and the protein and alginate on the other side, even if a thinner methyl cellulose gel is used, which might become necessary in order to make the emulsions more flowable/processable for continuous or semi-continuous processes.

### Experiment 6: Final Hardness and Hardening Time in Dependence of Alginate and Protein-Content, Without or With Methyl Cellulose

To support these observations, a separate multi-parameter Design of Experiment (DoE) was performed with 30 test groups with spherical particles of 25 mm diameter, produced according to the protocol 1.1-1.5. For this an emulsion with an oil content of 9 wt % (here rapeseed oil was used), with variation of contents of PPI, sodium alginate, methyl cellulose was prepared and precipitated by using an aqueous solution of CaCl 2-dihydrate with concentrations in the range of 2-4 wt % as a precipitation fluid and hardening at room temperature (20° C.):
Pea Protein Isolate (11.4-14 wt %)
Alginate (1.0-2 wt %)
CaCl₂-dihydrate-solution (2-4 wt %)
Methyl cellulose (0.2-0.8 wt %)
Temperature of measurement (=incubation temperature)

In the experiments, methyl cellulose was used in pre-hydrated form by providing a 2% solution of MC with shear-mixing at 5° C.

The data obtained in these experiments were used to calculate regressions curves for the relations of final hardness respectively hardening time for examples of samples with 11.4 and 14 wt % PPI in the emulsion and 3 wt % CaCl₂ in the precipitation fluid. Exemplary results at 14 wt % PPI for varying methyl cellulose-additions in combination with varied alginate and protein-contents are shown in FIGS. 6a) for final hardness and 6b) hardening time. In the FIGS. 6a and 6b the following abbreviations were used.
2%=2 wt % alginate
1.5%=1.5 wt % alginate
15 1%=1 wt % alginate
F_{f}[N]=Final hardness
tₕ [h]=Hardening time
wₘ [%]=mass fraction methyl cellulose [wt %]

Results show, for all PPI-concentrations, that at high alginate contents increasing amounts of methyl cellulose soften the fibres but when reducing alginate contents it increases the final hardness. Hardening time only slightly decreases at all concentrations.

Resulting from these comparisons, FIG. 7 is a contour plot showing also the correlated effect from alginate and PPI on the final hardness F_{f} given in Newton. The corresponding parameters are based on the center points from the same DoE carried out for experiment 6: 3 wt % CaCl₂*2 H₂O, 0.5 wt % methyl cellulose, 1.5 wt % alginate and 12.8 wt % PPI. Here it can be observed that the loss in final hardness resulting from the decreasing fraction of alginate can be compensated by increasing the PPI content.

FIG. 7 is a contour plot showing the correlation of alginate and PPI on the final hardness. Final hardness: The corresponding parameters are based on the center points from the DoE (3 wt % CaCl₂*2 H₂O, 0.5 wt % methyl cellulose, 1.5 wt % alginate and 12.8 wt % PPI). In FIG. 7 the following abbreviations are used:
x-axis: amount of pea protein isolate in the emulsion PPI (wt %)
y-axis: amount of alginate in the emulsion Al (wt %)
Final hardness F_{f}[N]

### Experiment 7: Firmness of Particles Depending on the Curing Time in the CaCl₂-Solution at Room Temperature

In a related experiment, particles produced according to the protocol 1.1-1.5 with 2 different protein-alginate ratios, i.e. either with 12.4% PPI and 2.8% alginate or with 14% PPI, 2% alginate and 0.5% of a pre-emulsified methyl cellulose, remained for graduated time intervals of 0.5-7 hours and 24 hours at 20° C. in a 3 wt % aqueous CaCl₂-solution. The recipes are given in the following table 3. Total hardness and time were measured directly at each point in time with the target to achieve a stable product, even if not equally hard. The results are given in table 4 and visualized in FIG. 8.

TABLE 3
Recipes

| | Trials 1-10 | Trials 11-20 |
|---|---|---|
| Water | 75.4 | 74.5 |
| Sunflower Oil | 9.0 | 9.0 |
| Pea protein isolate | 12.8 | 14.0 |
| Sodium alginate | 2.8 | 2.0 |
| Methyl cellulose* | — | 0.5 |

*as 2 wt % aqueous gel

TABLE 4
firmness

| | | F_{f} [g] | F_{f} [g] |
|---|---|---|---|
| Trial No. | Curing time [h] | trials 1-10 | trials 11-20 |
| 1/11 | 0 | 515 | 394 |
| 2/12 | 1 | 789 | 592 |
| 3/13 | 1.5 | 956 | 804 |
| 4/14 | 2 | 1051 | 975 |
| 5/15 | 3 | 1130 | 977 |
| 6/16 | 4 | 1165 | 721 |
| 7/17 | 5 | 1151 | 857 |
| 8/18 | 6 | 1231 | 922 |
| 9/19 | 7 | 1274 | 964 |
| 10/20 | 24 | 1510 | 1045 |

Firmness of the particles depends on the curing/dwell time in the aqueous CaCl₂-dihydrate solution. At any point in time, firmness of the particles with reduced alginate, but with methyl cellulose was lower than the standard, but sufficient hardness (˜80-90% of the standard value) could be achieved by increased dwelling time in the curing solution, as shown in FIG. 8. In FIG. 8, the following abbreviations are used
T [h]=Curing time in CaCl₂ solution [h]
●=F_{f}[g] =Firmness trials 1-10
. . . . .=Log. Firmness trials 1-10 (trend line)
x=F_{f}[g]=Firmness trials 11-20
- - - - - 32 Log. Firmness trials 11-20 (trend line)

### Experiment 8: Mimicking Typical Hot Consumption Temperature of Finished Meat Substitute Products

In a further series of trials protein particles were produced according to the protocol 1.1-1.5 with hardening in a 3 wt % aqueous CaCl₂-dihydrate-solution and diffusion-incubation for 2 or 6 hours either at 20 or 70° C. with subsequent dry curing at 20° C. Hardness was measures at 70° C. to mimic typical consumption temperature of a finished meat substitute product. The emulsion either contained 12.4 wt % PPI and 2.8 wt % alginate (series a) or 14 wt % PPI, 2 wt % alginate and 0.5 wt % of a pre-gelled methyl cellulose (series b). The results for series a) are shown in FIGS. 9 and 11 and for series b) in FIGS. 10 and 12.

In the FIGS. 9-12 the following abbreviations are used:
#=no. of trial
F [g]=Hardness at point of measurement
for FIGS. 9-10:
cu 20 or 70° C., Fm 70° C.=cured at 20 or 70° C., measurement at 70° C.
cu 20 or 70° C. 2 h=cured at 20 or 70° C. for 2 h, then stored outside solution till 24 h
cu 20 or 70° C. 6 h=cured at 20 or 70° C. for 6 h, then stored outside solution till 24 h
cu 20° C. 24 h=cured at 20° C. for 24 h, measurement at 70° C.
mi=measured immediately
m24 h=measured after 24 h
for FIGS. 11-12:
cu 20° C., Fm 20° C. or 70° C.=cured at 20° C., measurement at 20 or 70° C.
cu 20° C. 2 h, 6 h, 24 h=cured at 20° C. for 2 or 6 h, then stored outside solution till 24 h, or fully cured at 20° C. for 24 h
m24 h 20 or 70° C.=measured after 24 h at 20 or 70° C.

Particles are in general softer for shorter curing time of 2 hours versus longer curing time of 6 hours for both curing temperatures and for both compositions, when immediately measured (columns 1, 3, 5, 7; 10, 12, 14, 16 in FIGS. 9 and 10), but hardness further increases after further rest period outside the curing solution up to 24 h (columns 2, 4, 6, 8; 11, 13, 15, 17 in FIGS. 9 and 10). Then differences between previously curing for 2 or 6 hours became smaller or more balanced, respectively. Additional comparisons are made for both compositions versus fibres fully cured for 24 h at 20° C. (columns 9 resp. 18 in FIGS. 9 and 10), for which hardnesses are somewhat higher, which can be explained by more calcium absorption.

Hardness after hardening at 70° C. vs. 20° C. was by trend higher after 2 hours since calcium uptake and contents of the fibres incubated at 70° C. were higher compared to the calcium contents of the fibres which were incubated at 20° C., but fibres hardened for 6 h in the calcium solution were harder after additional storage outside the solution compared to 2 hours hardening in the solution accounting for a totally higher calcium uptake after 6 h.

Particles with reduced alginate-content but with incorporated methyl cellulose were less hard than the standard fibres at all treatments. Longer curing times seem to reduce such differences.

However, in a comparison of the particles cured for 24 h at 20° C., but measured at 70° C. (columns 9 resp. 18 in FIGS. 11 and 12) with fibres cured in the calcium solution at 20° C. for 2, 6 and 24 h, and then measured at 20° C. after 24 h (columns 19-21 and 22-24 in FIGS. 11 and 12), the hot measured fibres (9 resp. 18 in FIGS. 11 and 12; mimicking mouthfeel at consumption) showed just slightly lower hardness.

The results also show that a generally observed loss of hardness of the fibres when heated for hot consumption can be reduced by the addition of methyl cellulose, which modifies the hardness of the particles and particularly increases the thermal stability of the fibre and thus improves mouthfeel at hot consumption and better preserves the texture.

Typically, products are more solid when cold than when they are hot. So if the hardness difference of a hot measured fibre is only slightly lower than a cold measured fibre, it means that the balanced compositions are very stable, both with a high alginate content and with a reduced alginate content and, on the other hand, increased protein content and methyl cellulose addition.

Hardening with shorter curing times is better at high temperatures, and accordingly also the hot strength compared to a fibre cured in the same short time at room temperature.

The same applies in principle to lower alginate and higher protein contents with methyl cellulose, which solidifies when hot.

### Production Example 1

Step 1: 756.7 g water at a temperature of 70-90° C. were added into a mixing vessel equipped with rotating knife blades (like bowl choppers, cutters, Stephan cutters, high speed emulsifiers, in particular those based on the rotor-stator principle, colloid mills and combinations thereof with a blender).
Step 2: 128.3 g of pea protein isolate, 20.0 g sodium alginate and 5 g methyl cellulose and 90 g of a vegetable fat or oil (sunflower or canola oil or any other vegetable oil/fat) were added and the total mass was mixed under shearing at 3000-5000 rpm for 10 minutes until a stable emulsion was achieved, whilst keeping the temperature at 70-90° C.
Step 3:A solution was prepared containing 3 wt % calcium chloride-dihydrate in water at 5-10° C.
Step 4:The emulsion was transferred into a first vessel, containing a sufficient amount of the solution made up in step 3, by pressing the emulsion through a grid in order to achieve a uniform, not too big particle diameter of about 25 mm. Instead of a grid, a perforated plate or a diaphragm knife can be used. The particles were precipitated/coagulated for 5 min. under stirring at 100-1000 rpm while keeping the temperature at 5-10° C. The amount of solution was sufficient to cover the particles. During this period a skin was formed on the surface of particles, whereby the particles became mechanically stable but did not completely harden.
Step 5:Then the particles were taken out of the solution and transferred into a separate vessel containing a cold (5-10° C.) 3 wt % aqueous solution of calcium chloride-dihydrate in an amount sufficient to cover the particles (in a volume ratio of about 1:1 compared to the emulsion), optionally with gentle stirring and keeping the temperature of the solution at 5-10° C., in order to generate complete uniform fibre formation.
Step 6:After a typical hardening time of 12 to 20 h the fibres were taken out of the solution and rinsed with fresh water in order to remove any curing solution from the surface of the particles. Then, the particles were dewatered on a vibrating sieve or in a centrifuge or similar. Thereafter the particles were cooled or frozen for storing before they are further processed.

### Production Example 2

The example was carried out as described for example 1 with the exception that the solution prepared in step 3 and step 4 and 5 were carried out at 72° C. Then the hardening time was in the range 6-12 h.

### Production Example 3

Step 1: 5 g of methyl cellulose were mixed with 245 g water and ice at a temperature of 5° C. under shearing in order to reach complete hydration.
Step 2: 511.7 g water at a temperature of 70-90° C. were added into a mixing vessel equipped with rotating knife blades (like bowl choppers, cutters, Stephan cutters, high speed emulsifiers, in particular those based on the rotor-stator principle, colloid mills and combinations thereof with a blender).
Step 3: 128.3 g of pea protein isolate and 20.0 g sodium alginate and 90 g of a vegetable fat or oil (sunflower or canola oil or any other vegetable oil/fat) were added to the mixture of step 2.
Step 4: 250 g of the pre-hydrated methyl cellulose solution of step 1 were added to the mass composed of steps 2 to 3 and the total mass was mixed under shearing at 3000-5000 rpm for 10 minutes until a stable emulsion was achieved, whilst keeping the temperature at 70-90° C.
Step 5:A solution was prepared containing 3 wt % calcium chloride-dihydrate in water at 72° C.
Step 6:The emulsion was transferred into a first vessel, containing a sufficient amount of the solution made up in step 5, by pressing the emulsion through a grid in order to achieve a uniform, not too big particle diameter of about 25 mm. Instead of a grid, a perforated plate or a diaphragm knife can be used. The particles were precipitated/coagulated for 5 min. under stirring at 100-1000 rpm while keeping the temperature at 72° C. The amount of solution was sufficient to cover the particles. During this period a skin was formed on the surface of particles, whereby the particles became mechanically stable but did not completely harden.
Step 7:Then the particles were taken out of the solution and were transferred into a separate vessel containing a warm (72° C.) 3 wt % aqueous solution of calciumchloride-dihydrate in an amount sufficient to cover the particles (in a volume ratio of about 1:1 compared to the emulsion), optionally with gentle stirring and keeping the temperature of the solution at 72° C., in order to generate complete uniform fibre formation.
Step 8:After the desired hardening time (typically 6 to 12 h) the fibres were taken out of the solution and rinsed with fresh water in order to remove any curing solution from the surface of the particles. Then, the particles were dewatered on a vibrating sieve or in a centrifuge or similar. Thereafter the particles were cooled or frozen for storing before they are further processed. The obtained protein product was more compact than the product obtained in production example 2.

The particles obtained in step 6 of example 1 or correspondingly of example 2 or in step 8 of example 2, respectively, can be processed to an artificial meat product by a process with comprises mixing the particles with binders of non-animal origin, such as hydrocolloids or plant fibres, and/or with herbs and spices, followed by shaping them to the desired shapes e.g. by using moulds or casings. The thus obtained shaped meat substitute products can be portioned, optionally coated, e.g. with batters, breadcrumbs or external seasonings. Then the products are chilled, frozen or pasteurized and packaged for distribution as finished meat substitute products such as burgers, nuggets, fish fingers, schnitzels, sausages and the like.

1. A process for preparing a vegan edible product from edible non-animal proteins, which comprises
(i) providing a malleable mass by mixing the following components
a) 7 to 20% by weight, based on the total weight of the malleable mass, of an edible protein component A, which is selected from the group consisting of edible vegetable protein materials, microbial protein materials and mixtures thereof,
b) 1 to 3.3% by weight, based on the total weight of the malleable mass, of a water-soluble organic polymeric gelling agent which is capable of being gelled by calcium ions as a component B, which is a water-soluble polysaccharide bearing carboxyl groups or a water soluble salt thereof,
c) optionally 0.05 to 1% by weight, based on the total weight of the malleable mass, of a water-swellable nonionic polysaccharide as a component C and
d) 1 to 15% by weight, based on the total weight of the malleable mass of an edible fat or oil of plant origin as a component D,
e) water ad 100% by weight;
(ii) comminuting the malleable mass into particles and
(iii) bringing the particles into contact with an aqueous solution of a calcium salt to achieve a hardening of the particle, where step (iii) is carried out simultaneously with step (ii) or after step (ii).

2. The process of claim 1, wherein the total amount of component B and component C is in the range of 1.0 to 3.4% by weight, in particular in the range of 1.4 to 2.8% by weight, based on the total weight of the malleable mass.

3. The process of any one of the preceding claims, wherein the mass percentage amounts of the components A, B and C is such that the following equation (I) is fulfilled:
X=a*A+b*B+c*C (I)
where [A], [B] and [C] are the mass percentages of components A, B and C, respectively, where
a represents a number in the range of 2.5 to 5,
b represents a number in the range of 10 to 25,
c represents a number in the range of 10 to 100, and
where X represents a number in the range of 90 to 110.

4. The process of any one of the preceding claims, where the mass ratio of component A to component B is in the range of 2:1 to 20:1, the mass ratio of component A to component C is in the range of 14:1 to 140:1 and the mass ratio of component B to component C is in the range of 1.5:1 to 20:1.

5. The process of any one of the preceding claims, wherein the component B is selected from the water-soluble salts of alginic acid, pectins and mixtures thereof.

6. The process of any one of the preceding claims, wherein the component C is methylcellulose and is present in the malleable mass.

7. The process of claim 6, wherein the methylcellulose is provided in pre-hydrated form before it is mixed in step (i) with the other components of the malleable mass.

8. The process of claim 7, wherein the pre-hydrated methylcellulose is provided as a 0.1 to 5% by weight aqueous gel obtained by dissolving methylcellulose in water at a temperature of below 20° C. and shearing the solution.

9. The process of any one of the preceding claims, where component A comprises at least 90% by weight, based on the total weight of the component A, of at least one protein material selected from isolates and concentrates of chickpea protein, faba bean protein, lentil protein, lupine protein, mung bean protein, pea protein or soy protein and mixtures thereof.

10. The process of any one of the preceding claims, where step (ii) comprises passing the malleable mass through a grid or a perforated plate into the aqueous solution of the calcium salt.

11. The process of any one of claims 1 to 9 where step (ii) comprises comminuting the malleable mass in the presence of the aqueous solution of the calcium salt.

12. The process of any one of the preceding claims, where in step (ii) the aqueous solution of the calcium salt has a concentration of calcium in the range of 0.5 to 1.5% by weight, based on the total weight of the aqueous solution of the calcium salt.

13. The process of any one of the preceding claims, wherein the mass ratio of the aqueous solution of the calcium salt to the particles formed from the malleable mass is in the range of 1:3 to 3:1.

14. The process of any one of the preceding claims, where step (iii) is carried out at a temperature of at least 50° C., in particular in the range of 50 to 75° C.

15. A process for preparing a vegan artificial meat product which comprises producing an edible product from edible non-animal proteins by the process of any one of the preceding claims, followed by processing the edible product to an artificial meat product.
